(19) United States
(12) Patent Application Publication
(10) Pub. No.: US 2024/0050175 A1
(43) Pub. Date: Feb. 15, 2024

(54) SURGICAL ROBOT, ROBOTIC SURGICAL SYSTEM, AND CONTROL METHOD FOR SURGICAL ROBOT

(71) Applicants: KAWASAKI JUKOGYO KABUSHIKI KAISHA, Kobe-shi, Hyogo (JP); MEDICAROID CORPORATION, Kobe-shi, Hyogo (JP)

(72) Inventors: Yuji KISHIDA, Kobe-shi (JP); Jota IDA, Kobe-shi (JP)

(73) Assignees: KAWASAKI JUKOGYO KABUSHIKI KAISHA, Kobe-shi, Hyogo (JP); MEDICAROID CORPORATION, Kobe-shi, Hyogo (JP)

(21) Appl. No.: 18/259,082

(22) PCT Filed: Dec. 17, 2021
(86) PCT No.: PCT/JP2021/046754

§ 371 Date: Jun. 23, 2023

(30) Foreign Application Priority Data
Dec. 25, 2020 (JP) 2020-216192

(51) Int. Cl. A61B 34/35; A61B 50/13; A61B 34/20; B25J 9/16

(52) U.S. Cl. CPC A61B 34/35; A61B 50/13; A61B 34/20; B25J 9/1689; A61B 2034/302

(57) ABSTRACT

A surgical robot (1) includes a controller (31) configured or programmed to perform a control to move a medical cart (3), to align a manipulator arm (60) with a position corresponding to a port (PT) or a trocar (T) provided on a body surface (S) based on an image captured by an imager (51).

### TECHNICAL FIELD

The present disclosure relates to a surgical robot, a robotic surgical system, and a control method for a surgical robot.

### BACKGROUND ART

Conventionally, a surgical robot aligned with a patient is known. Such a surgical robot is disclosed in Japanese Translation of PCT International Application Publication No 2017-515522, for example.

Japanese Translation of PCT International Application Publication No 2017-515522 discloses a teleoperational assembly (surgical robot) including a plurality of arms to which surgical instruments such as endoscopes are attached. The teleoperational assembly includes a base and a telescoping support column attached to the base. The telescoping support column is provided along a vertical direction. A telescoping boom is provided to extend in a horizontal direction from the telescoping support column. The plurality of arms are attached to an orienting platform provided at the tip end of the telescoping boom via a plurality of support beams.

Japanese Translation of PCT International Application Publication No 2017-515522 discloses that a reference laser line is emitted from the teleoperational assembly to a patient placed on an operating table. An operator such as a nurse or a technician who moves the teleoperational assembly moves the entire teleoperational assembly such that the plurality of arms are arranged along the reference laser line directly emitted to the patient according to guided setup prompts and audio and voice prompts appearing on a touchpad. This seeks to align the teleoperational assembly with the patient.

### PRIOR ART

### Patent Document

Patent Document 1: Japanese Translation of PCT International Application Publication No 2017-515522

### SUMMARY OF THE INVENTION

However, in the teleoperational assembly described in Japanese Translation of PCT International Application Publication No 2017-515522, the operator needs to align the teleoperational assembly with the patient, and the burden on the operator is increased.

The present disclosure is intended to solve the above problem. The present disclosure aims to provide a surgical robot, a robotic surgical system, and a control method for a surgical robot each capable of reducing the burden on an operator when a surgical robot is aligned.

In order to attain the aforementioned object, a surgical robot according to a first aspect of the present disclosure includes a robot main body including a manipulator arm to which a surgical instrument is attached, a medical cart to move the robot main body, a medical cart drive to drive the medical cart, an imager provided on the robot main body to image at least one of a surgical table and a patient placed on the surgical table, and a controller configured or programmed to perform at least one of a control to move the medical cart by the medical cart drive and a control to move the manipulator arm by the robot main body, to align the manipulator arm with a position corresponding to a port or a trocar, which is provided on a body surface of the patient placed on the surgical table and into which the surgical instrument is to be inserted, based on an image captured by the imager.

As described above, the surgical robot according to the first aspect of the present disclosure includes the controller configured or programmed to perform at least one of the control to move the medical cart by the medical cart drive and the control to move the manipulator arm by the robot main body, to align the manipulator arm with the position corresponding to the port or the trocar, which is provided on the body surface of the patient placed on the surgical table and into which the surgical instrument is to be inserted, based on the image captured by the imager. Accordingly, the manipulator arm is automatically aligned with the position corresponding to the port or the trocar by at least one of the control to move the medical cart by the medical cart drive and the control to move the manipulator arm by the robot main body, and thus when the surgical robot is aligned, the burden on an operator can be reduced.

A robotic surgical system according to a second aspect of the present disclosure is operable to support robotic surgery using a surgical robot, and includes the surgical robot, a processor, and a controller. The surgical robot includes a robot main body including a manipulator arm to which a surgical instrument is attached, a medical cart to move the robot main body, a medical cart drive to drive the medical cart, and an imager provided on the robot main body to image at least one of a surgical table and a patient placed on the surgical table. The processor is operable to plan a position of the manipulator arm with respect to a position corresponding to a port, which is provided on a body surface of the patient placed on the surgical table and into which the surgical instrument is to be inserted. The controller is configured or programmed to acquire an image captured by the imager, and perform at least one of a control to move the medical cart by the medical cart drive and a control to move the manipulator arm by the robot main body, to arrange the manipulator arm at the planned position based on the image.

In the robotic surgical system according to the second aspect of the present disclosure, as described above, the controller is configured or programmed to acquire the image captured by the imager, and perform at least one of the control to move the medical cart by the medical cart drive and the control to move the manipulator arm by the robot main body, to arrange the manipulator arm at the planned position based on the image. Accordingly, the manipulator arm is automatically arranged at the position planned with respect to the position corresponding to the port by performing at least one of the control to move the medical cart by the medical cart drive and the control to move the manipulator arm by the robot main body, and thus it is possible to provide the robotic surgical system capable of reducing the burden on an operator when the surgical robot is aligned.

A control method for a surgical robot including a robot main body including a manipulator arm to which a surgical instrument is attached, a medical cart to move the robot main body, and a medical cart drive to drive the medical cart according to a third aspect of the present disclosure includes imaging at least one of a surgical table and a patient placed on the surgical table by an imager provided on the robot main body, and performing at least one of a control to move the medical cart by the medical cart drive and a control to move the manipulator arm by the robot main body, to align the manipulator arm with a position corresponding to a port or a trocar, which is provided on a body surface of the patient placed on the surgical table and into which the surgical instrument is to be inserted, based on an image captured by the imager.

As described above, the control method for the surgical robot according to the third aspect of the present disclosure includes performing at least one of the control to move the medical cart by the medical cart drive and the control to move the manipulator arm by the robot main body, to align the manipulator arm with the position corresponding to the port or the trocar, which is provided on the body surface of the patient placed on the surgical table and into which the surgical instrument is to be inserted, based on the image captured by the imager. Accordingly, the manipulator arm is automatically aligned with the position corresponding to the port or the trocar by performing at least one of the control to move the medical cart by the medical cart drive and the control to move the manipulator arm by the robot main body, and thus it is possible to provide the control method for the surgical robot capable of reducing the burden on an operator when the surgical robot is aligned.

According to the present disclosure, as described above, it is possible to reduce the burden on the operator when the surgical robot is aligned.

### BRIEF DESCRIPTION OF THE DRAWINGS

FIG. 1 is a diagram showing the configuration of a surgical system according to a first embodiment of the present disclosure.

FIG. 2 is a diagram showing the configuration of a medical manipulator according to the first embodiment of the present disclosure.

FIG. 3 is a perspective view showing the configuration of a medical cart according to the first embodiment of the present disclosure.

FIG. 4 is a diagram showing the medical manipulator and a patient according to the first embodiment of the present disclosure.

FIG. 5 is a diagram showing marks displayed on a display.

FIG. 6 is a diagram showing trocars inserted into the body surface of the patient.

FIG. 7 is a diagram showing an endoscope.

FIG. 8 is a diagram showing a pivot position teaching instrument.

FIG. 9 is a diagram showing the configuration of a manipulator arm of the medical manipulator according to the first embodiment of the present disclosure.

FIG. 10 is a perspective view showing the configuration of an operation unit of the medical manipulator according to the first embodiment of the present disclosure.

FIG. 11 is a side view showing the configuration of the operation unit of the medical manipulator according to the first embodiment of the present disclosure.

FIG. 12 is a diagram showing the operation unit of the medical manipulator grasped by an operator according to the first embodiment of the present disclosure.

FIG. 13 is a diagram for illustrating translation of the manipulator arm.

FIG. 14 is a diagram for illustrating rotation of the manipulator arm.

FIG. 15 is a block diagram showing the configuration of a controller of the medical manipulator according to the first embodiment of the present disclosure.

FIG. 16 is a flowchart for illustrating a method for aligning the manipulator arm of the surgical robot according to the first embodiment of the present disclosure.

FIG. 17 is a diagram (1) showing the display of the medical cart according to the first embodiment of the present disclosure.

FIG. 18 is a diagram (2) showing the display of the medical cart according to the first embodiment of the present disclosure.

FIG. 19 is a diagram showing a state in which an imager is imaging a surgical table.

FIG. 20 is a diagram showing a state in which the imager is imaging the patient.

FIG. 21 is a diagram (1) showing alignment between the trocars and the marks displayed on the display.

FIG. 22 is a diagram (1) showing alignment between the trocars and the marks displayed on the display.

FIG. 23 is a side view showing the configuration of a surgical system according to a second embodiment of the present disclosure.

FIG. 24 is a flowchart for illustrating a method for planning the position of a manipulator arm of a surgical robot according to the second embodiment of the present disclosure.

FIG. 25 is a top view showing the configuration of the surgical system according to the second embodiment of the present disclosure.

FIG. 26 is a flowchart for illustrating a method for aligning the manipulator arm of the surgical robot according to the second embodiment of the present disclosure.

FIG. 27 is a side view (1) of a surgical robot according to a modified example.

FIG. 28 is a diagram showing identifiers according to a modified example.

FIG. 29 is a side view (2) of a surgical robot according to a modified example.

### MODES FOR CARRYING OUT THE INVENTION

Embodiments embodying the present disclosure are hereinafter described on the basis of the drawings.

### First Embodiment

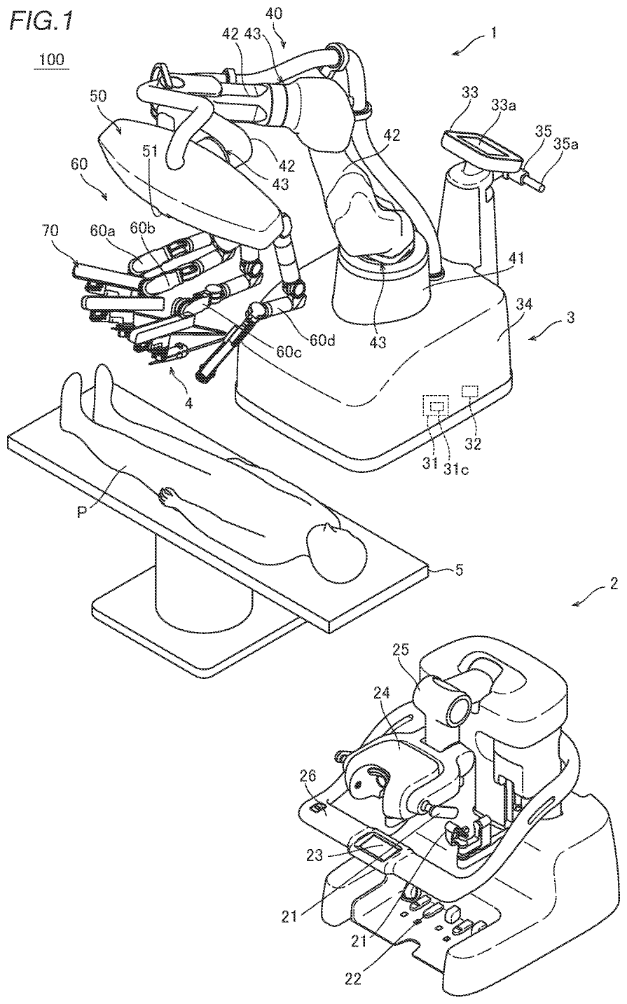

The configuration of a surgical system 100 according to a first embodiment is now described with reference to FIGS. 1 to 22. The surgical system 100 includes a medical manipulator 1 that is a patient P-side apparatus and a remote control apparatus 2 that is an operator-side apparatus to operate the medical manipulator 1. The medical manipulator 1 includes a medical cart 3 that moves a positioner 40, an arm base 50, and a plurality of manipulator arms 60, which are described below, and is movable. The remote control apparatus 2 is spaced apart from the medical manipulator 1, and the medical manipulator 1 is remotely operated by the remote control apparatus 2. A surgeon inputs a command to the remote control apparatus 2 to cause the medical manipulator 1 to perform a desired operation. The remote control apparatus 2 transmits the input command to the medical manipulator 1. The medical manipulator 1 operates based on the received command. The medical manipulator 1 is arranged in an operating room that is a sterilized sterile field. The medical manipulator 1 is an example of a “surgical robot”. The arm base 50 is an example of a robot main body.

The remote control apparatus 2 is arranged inside or outside the operating room, for example. The remote control apparatus 2 includes operation manipulator arms 21, operation pedals 22, a touch panel 23, a monitor 24, a support arm 25, and a support bar 26. The operation manipulator arms 21 include operation handles for the surgeon to input commands. The monitor 24 is a scope-type display that displays an image captured by an endoscope 6. The support arm 25 supports the monitor 24 so as to align the height of the monitor 24 with the height of the surgeon's face. The touch panel 23 is arranged on the support bar 26. The surgeon's head is detected by a sensor (not shown) provided in the vicinity of the monitor 24 such that the medical manipulator 1 can be operated by the remote control apparatus 2. The surgeon operates the operation manipulator arms 21 and the operation pedals 22 while visually recognizing an affected area on the monitor 24. Thus, a command is input to the remote control apparatus 2. The command input to the remote control apparatus 2 is transmitted to the medical manipulator 1.

The medical cart 3 includes a controller 31 that controls the operation of the medical manipulator 1 and a storage 32 that stores programs or the like to control the operation of the medical manipulator 1. The controller 31 of the medical cart 3 controls the operation of the medical manipulator 1 based on the command input to the remote control apparatus 2.

The medical cart 3 includes an input 33. The input 33 receives operations to move the positioner 40, the arm base 50, and the plurality of manipulator arms 60 or change their postures mainly in order to prepare for surgery before the surgery.

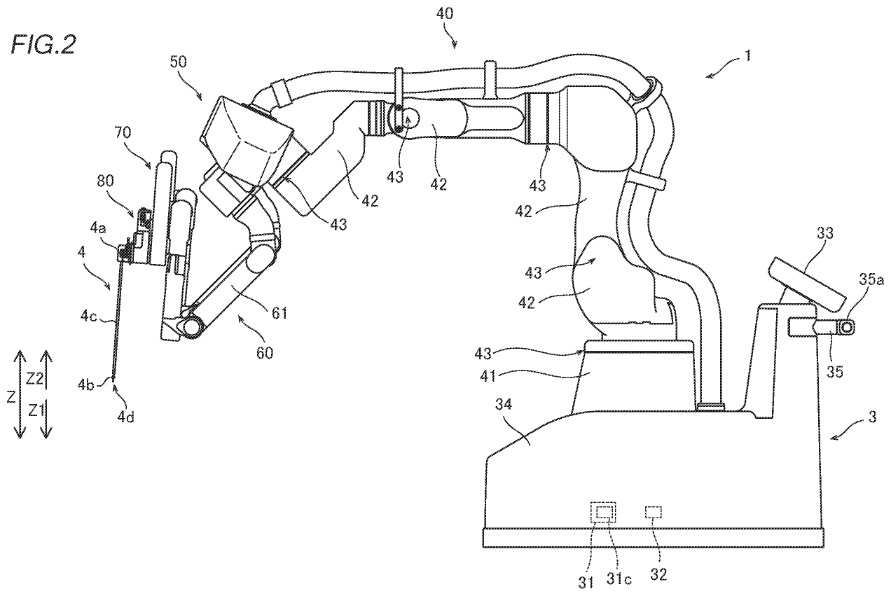

As shown in FIGS. 1 and 2, the medical manipulator 1 is arranged in the operating room. The medical manipulator 1 includes the medical cart 3, the positioner 40, the arm base 50, and the plurality of manipulator arms 60. The arm base 50 is attached to the tip end of the positioner 40. The arm base 50 has a relatively long rod shape. The bases of the plurality of manipulator arms 60 are attached to the arm base 50. Each of the plurality of manipulator arms 60 is able to take a stored posture that is a folded posture. The arm base 50 and the plurality of manipulator arms 60 are covered with sterile drapes (not shown) and used.

In the first embodiment, the arm base 50 includes an imager 51. The imager 51 images at least one of a surgical table 5 and a patient P placed on the surgical table 5.

The positioner 40 includes a 7-axis articulated robot, for example. The positioner 40 is arranged on a casing 34 of the medical cart 3. The positioner 40 moves the arm base 50. Specifically, the positioner 40 moves the position of the arm base 50 three-dimensionally.

The positioner 40 includes a base 41 and a plurality of links 42 coupled to the base 41. The plurality of links 42 are coupled to each other by joints 43.

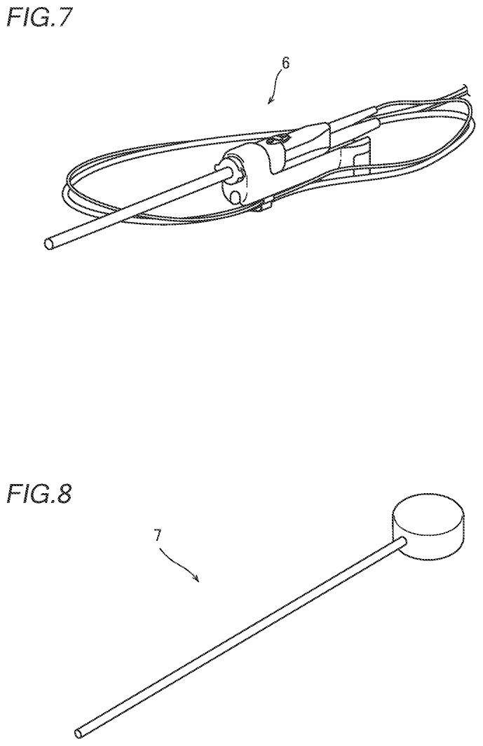

As shown in FIG. 1, a surgical instrument 4 is attached to the tip end of each of the plurality of manipulator arms 60. The surgical instrument 4 includes a replaceable instrument (see FIG. 2) or the endoscope 6 (see FIG. 7), for example. When a pivot position described below is taught, a pivot position teaching instrument (see FIG. 8) is attached instead of the instrument to the tip end of the manipulator arm 60 to which the instrument is to be attached.

As shown in FIG. 2, the instrument as the surgical instrument 4 includes a driven unit 4a driven by servomotors M2 provided in a holder 71 of each of the manipulator arms 60. An end effector 4b is provided at the tip end of the instrument. The end effector 4b includes a pair of forceps, a pair of scissors, a grasper, a needle holder, a microdissector, a stable applier, a tacker, a suction cleaning tool, a snare wire, a clip applier, etc. as instruments having joints. The end effector 4b includes a cutting blade, a cautery probe, a washer, a catheter, a suction orifice, etc. as instruments having no joint. The surgical instrument 4 includes a shaft 4c that connects the driven unit 4a to the end effector 4b. The driven unit 4a, the shaft 4c, and the end effector 4b are arranged along a Z direction.

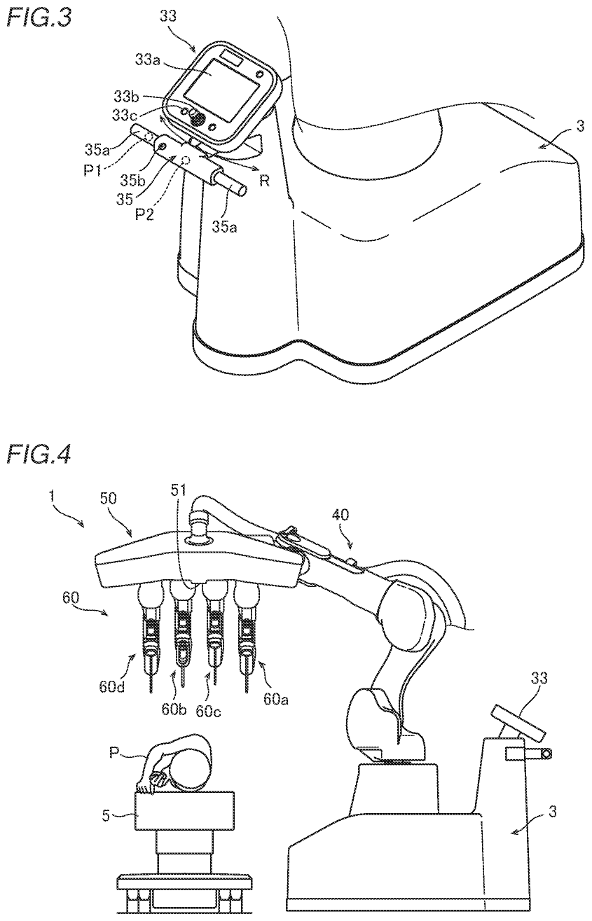

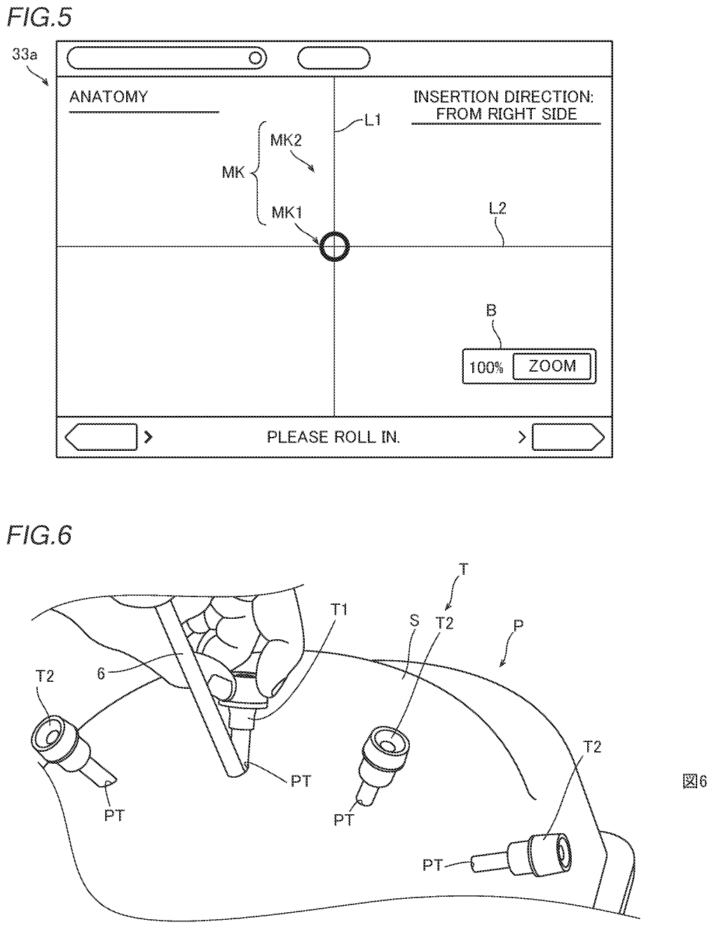

As shown in FIGS. 3 and 4, the input 33 of the medical cart 3 includes a display 33a. The display 33a displays the patient P imaged by the imager 51 in real time. As shown in FIG. 5, on the display 33a, the image of the patient P captured by the imager 51 and marks MK for aligning the arm base 50 with the patient P are superimposed and displayed. Specifically, on the display 33a, an image of trocars T (see FIG. 6) for inserting the endoscope 6 from the body surface S of the patient P captured the imager 51 and the marks MK for aligning the arm base 50 with the trocars T are superimposed and displayed. The marks MK displayed on the display 33a are aligned, on the display 33a, with the trocars T displayed on the display 33a such that the manipulator arms 60 are aligned with a surgical location in the patient P placed on the surgical table 5. The alignment of the manipulator arms 60 is manually performed by an operator such as a nurse or a technician, or automatically performed by the controller 31 as described below.

The image of the patient P and the trocars T displayed on the display 33a is an image actually captured by the imager 51, and the marks MK are graphical user interface (GUI) images generated by the controller 31 and are stored in the storage 32. The controller 31 is equipped with an image processing circuit 31c that displays an image obtained from the imager 51 on the display 33a. The image processing circuit 31c using a field programmable gate array (FPGA) mounted on the controller 31 synthesizes the image of the patient P and the trocars T actually captured by the imager 51 and stored in the storage 32 and the marks MK of the GUI images and displays the composite on the display 33a in real time with a delay that people do not recognize. The image processing circuit 31c may include an application specific integrated circuit (ASIC) or a system on a chip (SoC), for example, other than the field programmable gate array (FPGA).

Specifically, the trocars T include a first trocar T1 into which the endoscope 6 is inserted and second trocars T2 into which the surgical instruments 4 other than the endoscope 6 are inserted. The marks MK displayed on the display 33a include a first mark MK1 aligned with the first trocar T1 on the display 33a and a second mark MK2 aligned with the second trocars T2 on the display 33a. More specifically, the first mark MK1 is displayed on a substantially central portion of the display 33a and has a substantially circular shape. The second mark MK2 has a cross shape centered on the substantially circular first mark MK1.

The size of the substantially circular first mark MK1 is larger than the size of the first trocar T1 displayed on the display 33a. Specifically, the diameter of the substantially circular first mark MK1 is larger than the diameter of the first trocar T1 having a substantially circular cross-section.

A plurality of second trocars T2 are provided on the body surface S of the patient P. The plurality of second trocars T2 are arranged on a substantially straight line. The trocars T are arranged in the order of the second trocar T2, the first trocar T1, the second trocar T2, and the second trocar T2 so as to correspond to four manipulator arms 60.

The display 33a has a substantially rectangular shape. For example, the display 33a has a horizontally long rectangular shape as viewed from the operator. The cross-shaped second mark MK2 includes a substantially linear first line L1 provided along the longitudinal direction of the substantially rectangular display 33a, and a substantially linear second line L2 provided along the transverse direction of the substantially rectangular display 33a. The medical manipulator 1 is configured such that the plurality of second trocars T2 are aligned along the first line L1 or the second line L2 on the display 33a.

The display of the first line L1 and the second line L2 on the display 33a is fixed on the display 33a. The display 33a is fixed to the medical cart 3. Thus, a direction along the first line L1 corresponds to a forward-rearward direction that is the moving direction of the medical cart 3. The image of the patient P displayed on the display 33a changes with movement of the imager 51 of the arm base 50 or movement of the medical cart 3.

A magnification change button B is provided to enlarge or reduce an image of the first mark MK1 together with the image of the patient P displayed on the display 33a. The magnification change button B is displayed on a touch panel. When the magnification change button B is pressed, the magnification percentage of an image is changed in a loop of 100%, 200%, 400%, 100%, and 200% in this order. When the magnification percentage of the image is 100%, an end of the surgical table 5 and a nearby assistant and nurse are displayed on the display 33a.

The first trocar T1 and the first mark MK1 displayed on the display 33a are aligned with each other on the display 33a manually by the operator or automatically by the controller 31, and the second trocars T2 and the second mark MK2 displayed on the display 33a are aligned with each other on the display 33a manually by the operator or automatically by the controller 31 such that the manipulator arms 60 are aligned with the surgical location in the patient P placed on the surgical table 5. The details of alignment of the manipulator arms 60 with the surgical location in the patient P are described below.

As shown in FIG. 3, a joystick 33b for operating movement of the positioner 40 is provided in the vicinity of the display 33a of the medical cart 3. The positioner 40 can be operated three-dimensionally by selecting an operation mode displayed on the display 33a and operating the joystick 33b.

An enable switch 33c for enabling or disabling movement of the positioner 40 is provided in the vicinity of the joystick 33b of the medical cart 3. The joystick 33b is operated while the enable switch 33c is being pressed to enable movement of the positioner 40 such that the positioner 40 is moved.

A handle 35 is provided in the vicinity of the display 33a of the medical cart 3 to operate movement of the medical cart 3. The handle 35 includes a throttle 35a that is gripped and rotated by the operator such as a nurse or a technician to operate movement of the medical cart 3. As the handle 35 is rotated, the moving direction of the medical cart 3 is changed.

An enable switch 35b for enabling or disabling movement of the medical cart 3 is provided in the vicinity of the handle 35 of the medical cart 3. The throttle 35a of the handle 35 is operated while the enable switch 35b is being pressed to enable movement of the medical cart 3 such that the medical cart 3 is moved in the forward-rearward direction.

While the trocars T and the marks MK displayed on the display 33a are aligned with each other on the display 33a, the positioner 40 is controlled to move the arm base 50 in order for the imager 51 to image a region vertically therebelow. This control is performed by the controller 31 that controls the operation of the medical manipulator 1.

The configuration of the manipulator arms 60 is now described in detail.

As shown in FIG. 9, each of the manipulator arms 60 includes an arm portion 61 including a base 62, links 63 and joints 64, and a translation mechanism 70 provided at the tip end of the arm portion 61. The tip end sides of the manipulator arms 60 can be three-dimensionally moved with respect to the arm base 50 on the base sides of the manipulator arms 60. The plurality of manipulator arms 60 have the same configuration as each other.

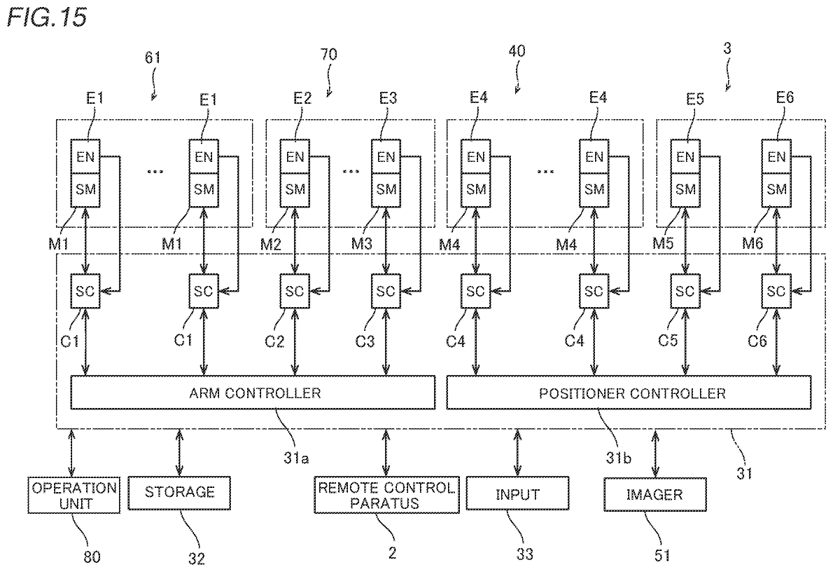

The translation mechanism 70 is provided on the tip end side of the arm portion 61, and the surgical instrument 4 is attached thereto. The translation mechanism 70 translates the surgical instrument 4 in a direction in which the surgical instrument 4 is inserted into the patient P. Furthermore, the translation mechanism 70 translates the surgical instrument 4 relative to the arm portion 61. Specifically, the translation mechanism 70 includes the holder 71 that holds the surgical instrument 4. The servomotors M2 (see FIG. 15) are housed in the holder 71. The servomotors M2 rotate rotary bodies provided in the driven unit 4a of the surgical instrument 4. The rotary bodies in the driven unit 4a are rotated such that the end effector 4b is operated.

The manipulator arms 60 are attachable to and detachable from the arm base 50.

The arm portion 61 includes a 7-axis articulated robot arm. The arm portion 61 includes the base 62 to attach the arm portion 61 to the arm base 50, and a plurality of links 63 coupled to the base 62. The plurality of links 63 are coupled to each other by the joints 64.

The translation mechanism 70 translates the surgical instrument 4 attached to the holder 71 along the Z direction (a direction in which the shaft 4c extends) by translating the holder 71 along the Z direction.

Specifically, the translation mechanism 70 includes a base end side link 72 connected to the tip end of the arm portion 61, a tip end side link 73, and a coupling link 74 provided between the base end side link 72 and the tip end side link 73. The holder 71 is provided on the tip end side link 73.

The coupling link 74 of the translation mechanism 70 is configured as a double speed mechanism that moves the tip end side link 73 relative to the base end side link 72 along the Z direction. The tip end side link 73 is moved along the Z direction relative to the base end side link 72 such that the surgical instrument 4 provided on the holder 71 is translated along the Z direction. The tip end of the arm portion 61 is connected to the base end side link 72 so as to rotate the base end side link 72 about a Y direction orthogonal to the Z direction.

As shown in FIG. 1, the endoscope 6 is attached to one (a manipulator arm 60b, for example) of the plurality of manipulator arms 60, and surgical instruments 4 other than the endoscope 6 are attached to the remaining manipulator arms 60 (manipulator arms 60a, 60c, and 60d, for example).

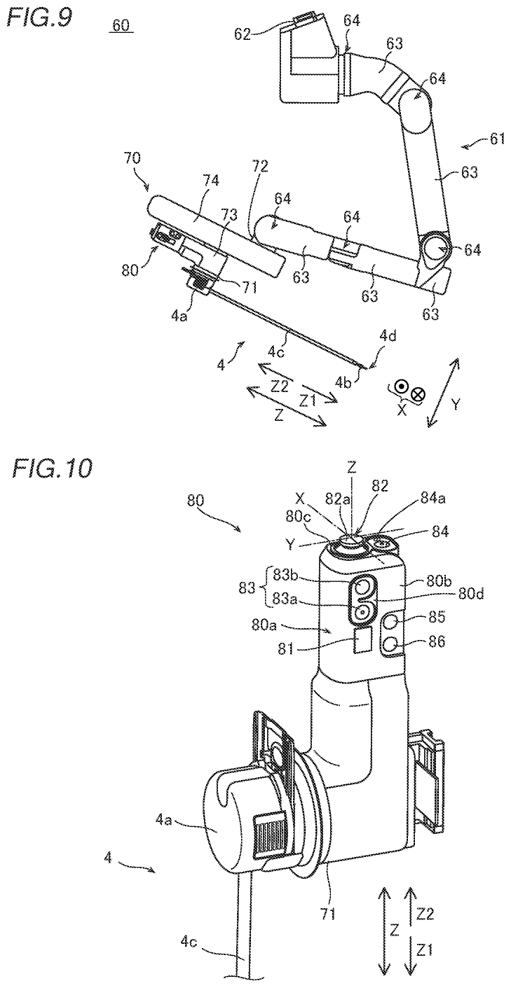

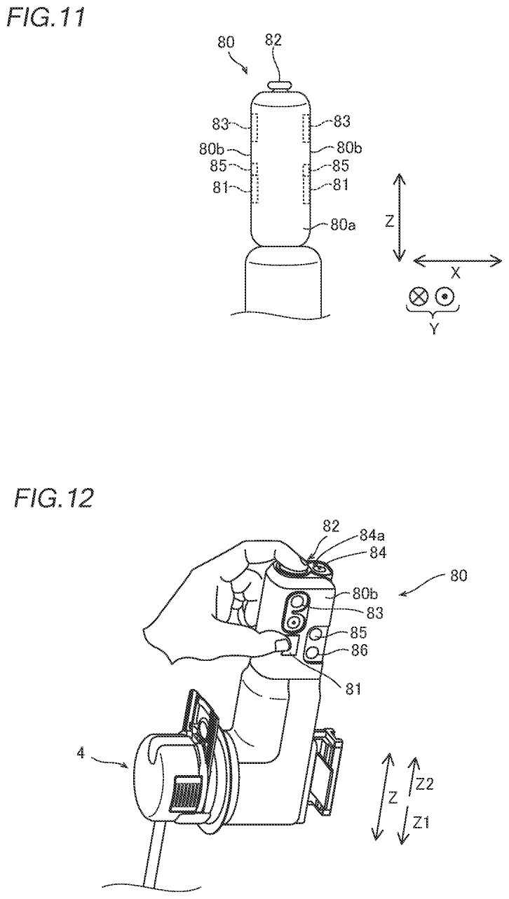

As shown in FIG. 10, the medical manipulator 1 includes an operation unit 80 attached to each of the manipulator arms 60 to operate the manipulator arm 60. The operation unit 80 includes enable switches 81, a joystick 82, and switch unit 83. The enable switches 81 enable or disable movement of the manipulator arm 60 in response to the joystick 82 and the switch units 83. The enable switches 81 enable movement of the surgical instrument 4 by the manipulator arm 60 when the enable switches 81 are pressed by an operator such as a nurse or an assistant grasping the operation unit 80. A pair of enable switches 81 are provided on opposite sides of the outer peripheral surface 80a of the operation unit 80 (see FIG. 11).

Specifically, the enable switches 81 are push-button switches that are pressed by the operator's fingers. The enable switches 81 are pressed such that it becomes possible to perform a control to energize servomotors M1 to M3. In other words, the enable switches 81 are pressed such that it becomes possible to perform a control to drive the servomotors M1 to M3. That is, it is possible to perform a control to move the manipulator arm 60 only while the enable switches 81 are being pressed.

As shown in FIG. 12, the operator tilts and operates the joystick 82 with their finger. The manipulator arm 60 is controlled to move according to a direction in which the joystick 82 is tilted and an angle at which the joystick 82 is tilted. The operator brings their finger into contact with the tip end 82a of the joystick 82, moves their finger, and tilts the joystick 82 to operate the joystick 82. Only while the enable switches 81 are being pressed, a signal input based on an operation on the joystick 82 is received. That is, while the enable switches 81 are not pressed, the manipulator arm 60 is not moved even when the joystick 82 is operated.

As shown in FIGS. 10 and 11, the joystick 82 is provided on an end face 80c of the operation unit 80 that intersects with the outer peripheral surface 80a. The operator can operate the joystick 82 with their finger while grasping the outer peripheral surface 80a of the operation unit 80 and pressing the enable switches 81 to enable movement of the manipulator arm 60. For example, as shown in FIG. 12, the operator operates the joystick 82 provided on the end face 80c of the operation unit 80 with their index finger, for example, while pressing the pair of enable switches 81 provided on the outer peripheral surface 80a of the operation unit 80 with their thumb and middle finger, for example. Thus, substantially constant distances between the operator's thumb and middle finger that grasp the operation unit 80 and the operator's index finger that operates the joystick 82 can be easily maintained. Which fingers are used to operate the enable switches 81 and the joystick 82 is not limited to the above example. Furthermore, movement of the manipulator arm 60 may be enabled while only one of the pair of enable switches 81 is being pressed.

With the joystick 82, movement of the surgical instrument 4 by the manipulator arm 60 is operated such that the tip end 4d (see FIG. 9) of the surgical instrument 4 moves on a predetermined plane. The operation unit 80 includes the switch units 83 for the operator to operate movement of the surgical instrument 4 by the manipulator arm 60 such that the tip end 4d of the surgical instrument 4 moves along the longitudinal direction of the surgical instrument 4 orthogonal to the predetermined plane. The predetermined plane on which the tip end 4d of the surgical instrument 4 moves refers to a plane (an X-Y plane in FIG. 10) parallel to the end face 80c of the operation unit 80. The longitudinal direction of the surgical instrument 4 orthogonal to the predetermined plane refers to the Z direction orthogonal to the X-Y plane in FIG. 10. Coordinates represented by an X-axis, a Y-axis, and a Z-axis in FIG. 10 are referred to as a tool coordinate system (or a base coordinate system). When the switch units 83 are pressed while the enable switches 81 are being pressed, the tip end 4d of the surgical instrument 4 is moved along the longitudinal direction of the surgical instrument 4.

Each of the switch units 83 includes a switch 83a to move the tip end 4d of the surgical instrument 4 in the direction in which the surgical instrument 4 is inserted into the patient P along the longitudinal direction of the surgical instrument 4, and a switch 83b to move the tip end 4d of the surgical instrument 4 in a direction opposite to the direction in which the surgical instrument 4 is inserted into the patient P. Both the switch 83a and the switch 83b are push-button switches. The switch units 83 are provided on the opposite sides of the outer peripheral surface 80a of the operation unit 80 (see FIG. 11). Specifically, each (a pair of switches 83a and 83b) of the switch units 83 is provided on each of opposite side surfaces of the operation unit 80.

As shown in FIG. 10, the operation unit 80 includes pivot buttons 85 to teach a pivot position PP that serves as a fulcrum (see FIG. 14) for movement of the surgical instrument 4 attached to the manipulator arm 60. The pivot buttons 85 are provided adjacent to the enable switches 81 on surfaces 80b of the operation unit 80. When the pivot buttons 85 are pressed, the pivot position PP is taught. A pair of pivot buttons 85 are provided on the opposite sides of the outer peripheral surface 80a of the operation unit 80 (see FIG. 11).

As shown in FIGS. 10 and 11, adjustment buttons 86 are provided on the surfaces 80b of the operation unit 80 to optimize the position of the manipulator arm 60. After the pivot position PP for the manipulator arm 60 to which the endoscope 6 has been attached is taught, the adjustment buttons 86 are pressed such that the positions of the other manipulator arms 60 (arm base 50) are optimized. A pair of adjustment buttons 86 are provided on the opposite sides of the outer peripheral surface 80a of the operation unit 80.

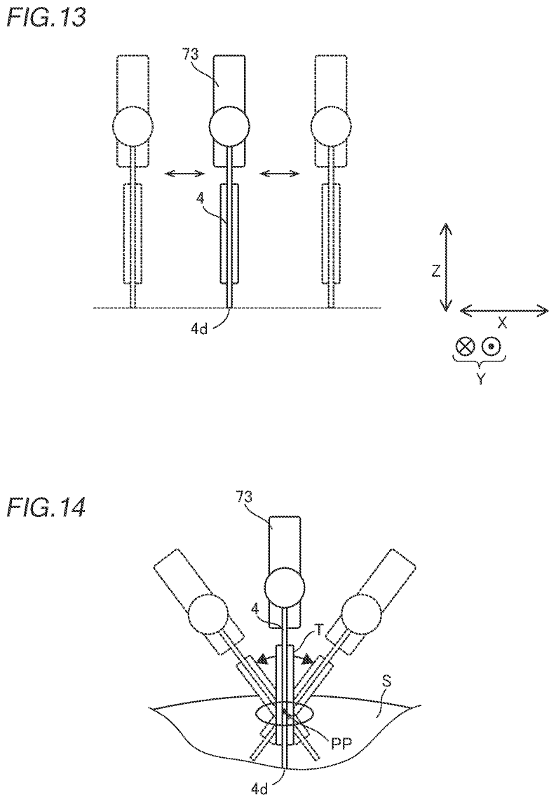

As shown in FIG. 10, the operation unit 80 includes a mode switching button 84 to switch between a mode for translating the surgical instrument 4 attached to the arm manipulator 60 (see FIG. 13) and a mode for rotationally moving the surgical instrument 4 (see FIG. 14). In the operation unit 80, the mode switching button 84 is arranged in the vicinity of the joystick 82.

Specifically, on the end face 80c of the operation unit 80, the mode switching button 84 is provided adjacent to the joystick 82. The mode switching button 84 is a push-button switch. Furthermore, a mode indicator 84a is provided in the vicinity of the mode switching button 84. The mode indicator 84a indicates a switched mode. Specifically, the mode indicator 84a is turned on (rotational movement mode) or off (translational mode) such that the current mode (translational mode or rotational movement mode) is indicated.

As shown in FIG. 13, in the mode for translating the manipulator arm 60, the manipulator arm 60 is moved such that the tip end 4d of the surgical instrument 4 moves on the X-Y plane. As shown in FIG. 14, in the mode for rotationally moving the manipulator arm 60, when the pivot position PP is not taught, the manipulator arm 60 is moved such that the surgical instrument 4 rotationally moves about the end effector 4b, and when the pivot position PP is taught, the manipulator arm 60 is moved such that the surgical instrument 4 rotationally moves about the pivot position PP as a fulcrum. The surgical instrument 4 is rotationally moved while the shaft 4c of the surgical instrument 4 is inserted into the trocar T.

As shown in FIG. 9, the operation unit 80 is provided on the translation mechanism 70. The operation unit 80 is attached to the translation mechanism 70 so as to be adjacent to the surgical instrument 4 attached to the translation mechanism 70. Specifically, the operation unit 80 is attached to the tip end side link 73 of the translation mechanism 70. The operation unit 80 is arranged adjacent to the driven unit 4a of the surgical instrument 4.

As shown in FIG. 15, the manipulator arm 60 includes a plurality of servomotors M1, encoders E1, and speed reducers (not shown) so as to correspond to a plurality of joints 64 of the arm portion 61. The encoders E1 detect the rotation angles of the servomotors M1. The speed reducers slow down rotation of the servomotors M1 to increase the torques.

As shown in FIG. 15, the translation mechanism 70 includes the servomotors M2 to rotate the rotary bodies provided in the driven unit 4a of the surgical instrument 4, the servomotor M3 to translate the surgical instrument 4, encoders E2, an encoder E3, and speed reducers (not shown). The encoders E2 and E3 detect the rotation angles of the servomotors M2 and M3, respectively. The speed reducers slow down rotation of the servomotors M2 and M3 to increase the torques.

The positioner 40 includes a plurality of servomotors M4, encoders E4, and speed reducers (not shown) so as to correspond to a plurality of joints 43 of the positioner 40. The encoders E4 detect the rotation angles of the servomotors M4. The speed reducers slow down rotation of the servomotors M4 to increase the torques.

The medical cart 3 includes servomotors M5 to drive a plurality of front wheels (not shown) of the medical cart 3, respectively, encoders E5, speed reducers (not shown), and brakes. The speed reducers slow down rotation of the servomotors M5 to increase the torques. Furthermore, a potentiometer P1 is provided on the throttle 35a of the medical cart 3, and the servomotors M5 of the front wheels are driven based on a rotation angle detected by the potentiometer P1 according to the twist of the throttle 35a. The rear wheels (not shown) of the medical cart 3 are of the dual wheel type, and the rear wheels are steered based on the rightward-leftward (R direction) rotation of the handle 35. Furthermore, a potentiometer P2 is provided on the handle 35 of the medical cart 3, and servomotors M6, encoders E6, and speed reducers (not shown) are provided on the rear wheels of the medical cart 3. The speed reducers slow down rotation of the servomotors M6 to increase the torques. The servomotors M6 are driven based on a rotation angle detected by the potentiometer P2 according to the rightward-leftward (R direction) rotation of the handle 35. That is, steering of the rear wheels by the rightward-leftward (R direction) rotation of the handle 35 is power-assisted by the servomotors M6. The servomotors M5 and the servomotors M6 are examples of a medical cart drive.

The front wheels of the medical cart 3 is driven such that the medical cart 3 moves in the forward-rearward direction. Furthermore, the handle 35 of the medical cart 3 is rotated such that the rear wheels are steered, and the medical cart 3 turns in a rightward-leftward direction.

The controller 31 of the medical cart 3 includes an arm controller 31a to control movement of the plurality of manipulator arms 60 based on commands, and a positioner controller 31b to control movement of the positioner 40, driving of the front wheels (not shown) of the medical cart 3, and steering driving of the rear wheels (not shown) of the medical cart 3 based on commands. Servo controllers C1 that control the servomotors M1 to drive the manipulator arm 60 are electrically connected to the arm controller 31a. The encoders E1 that detect the rotation angles of the servomotors M1 are electrically connected to the servo controllers C1.

Servo controllers C2 that control the servomotors M2 to drive the surgical instrument 4 are electrically connected to the arm controller 31a. The encoders E2 that detect the rotation angles of the servomotors M2 are electrically connected to the servo controllers C2. A servo controller C3 that controls the servomotor M3 to translate the translation mechanism 70 is electrically connected to the arm controller 31a. The encoder E3 that detects the rotation angle of the servomotor M3 is electrically connected to the servo controller C3.

An operation command input to the remote control apparatus 2 is input to the arm controller 31a. The arm controller 31a generates position commands based on the input operation command and the rotation angles detected by the encoders E1 (E2, E3), and outputs the position commands to the servo controllers C1 (C2, C3). The servo controllers C1 (C2, C3) generate torque commands based on the position commands input from the arm controller 31a and the rotation angles detected by the encoders E1 (E2, E3), and output the torque commands to the servomotors M1 (M2, M3). Thus, the manipulator arm 60 is moved according to the operation command input to the remote control apparatus 2.

The controller 31 (arm controller 31a) operates the manipulator arm 60 based on an input signal from the joystick 82 of the operation unit 80. Specifically, the arm controller 31a generates position commands based on the input signal (operation command) input from the joystick 82 and the rotation angles detected by the encoders E1, and outputs the position commands to the servo controllers C1. The servo controllers C1 generate torque commands based on the position commands input from the arm controller 31a and the rotation angles detected by the encoders E1, and output the torque commands to the servomotors M1. Thus, the manipulator arm 60 is moved according to the operation command input to the joystick 82.

The controller 31 (arm controller 31a) operates the manipulator arm 60 based on an input signal from each of the switch units 83 of the operation unit 80. Specifically, the arm controller 31a generates a position command based on the input signal (operation command) input from each of the switch units 83 and the rotation angle detected by the encoders E1 or the encoder E3, and outputs the position command to the servo controllers C1 or the servo controller C3. The servo controllers C1 or the servo controller C3 generates a torque command based on the position command input from the arm controller 31a and the rotation angle detected by the encoders E1 or the encoder E3, and outputs the torque command to the servomotors M1 or the servomotor M3. Thus, the manipulator arm 60 is moved according to the operation command input to each of the switch units 83.

As shown in FIG. 15, servo controllers C4 that control the servomotors M4 to move the positioner 40 are electrically connected to the positioner controller 31b. The encoders E4 that detect the rotation angles of the servomotors M4 are electrically connected to the servo controllers C4. Servo controllers C5 that control the servomotors M5 to drive the front wheels (not shown) of the medical cart 3 are electrically connected to the positioner controller 31b. The encoders E5 that detect the rotation angles of the servomotors M5 are electrically connected to the servo controllers C5.

An operation command related to setting a preparation position, for example, is input from the input 33 to the positioner controller 31b. The positioner controller 31b generates position commands based on the operation command input from the input 33 and the rotation angles detected by the encoders E4, and outputs the position commands to the servo controllers C4. The servo controllers C4 generate torque commands based on the position commands input from the positioner controller 31b and the rotation angles detected by the encoders E4, and output the torque commands to the servomotors M4. Thus, the positioner 40 is moved according to the operation command input to the input 33. Similarly, the positioner controller 31b moves the medical cart 3 based on the operation command from the input 33.

A control method for the medical manipulator 1 is now described. In the first embodiment, ports PT or the trocars T are provided in advance on the body surface S of the patient P placed on the surgical table 5.

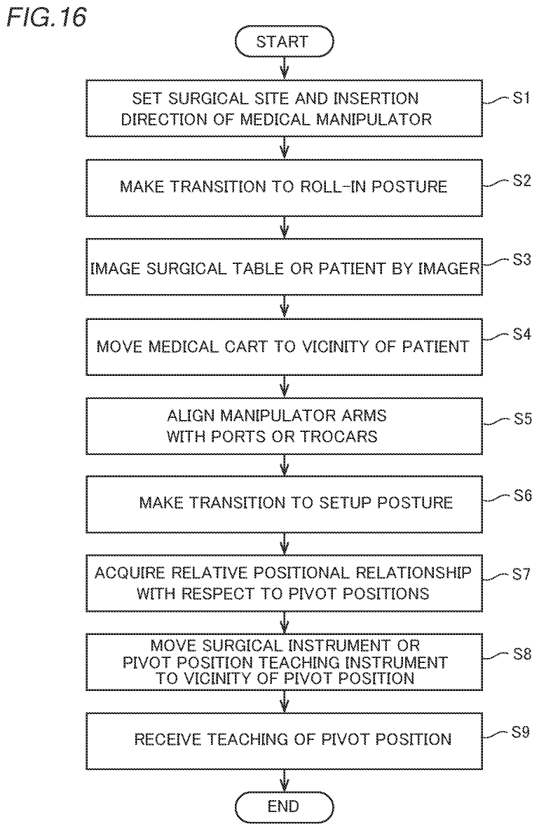

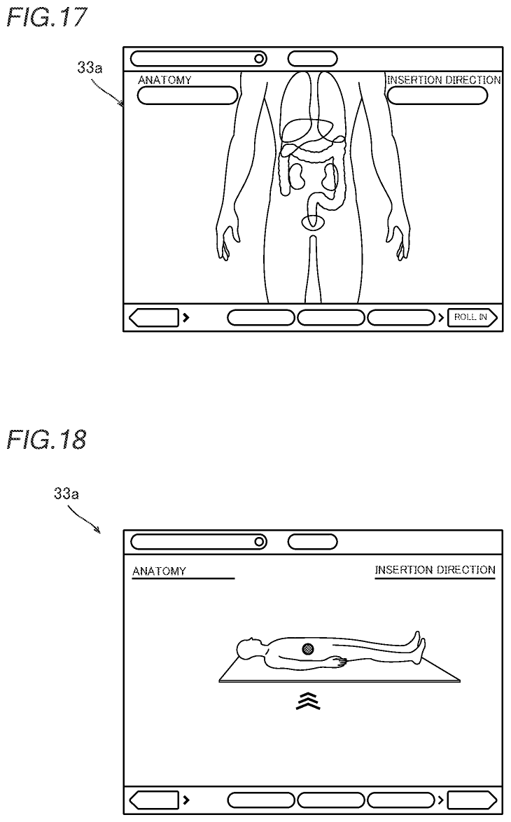

As shown in FIG. 16, first, in step S1, preparation for positioning the medical manipulator 1 is performed. Specifically, as shown in FIG. 17, on the touch panel of the display 33a, a surgical site (anatomy: “abdomen”, for example) and a direction in which the medical manipulator 1 is inserted into the patient P (“from the right side”, for example) are selected.

Then, in step S2, as shown in FIG. 17, a “roll in” button displayed on the display 33a is pressed. Thus, a roll-in mode is set, and the movement operation of the arm base 50 and the manipulator arms 60 is controlled such that the medical manipulator 1 takes a roll-in posture. The roll-in posture refers to a posture in which each manipulator arm 60 is folded so as not to interfere with the patient P when the manipulator arm 60 is positioned above the patient P by movement of the medical manipulator 1, a posture in which the arm base 50 is arranged by the positioner 40 such that the imager 51 provided on the arm base 50 can image the region vertically therebelow, and a posture in which the arm base 50 is arranged by the positioner 40 such that the orientation of the arm base 50 corresponds to the arrangement direction of each manipulator arm 60 determined based on the information on the surgical site and the information on the insertion direction selected in step S1. That is, after the “roll in” button displayed on the display 33a is pressed such that a transition to the roll-in mode is made, the joystick 33b is operated while the enable switch 33c is being pressed to enable movement of the positioner 40 such that the controller 31 moves the positioner 40 and each manipulator arm 60 such that the medical manipulator 1 automatically takes the roll-in posture.

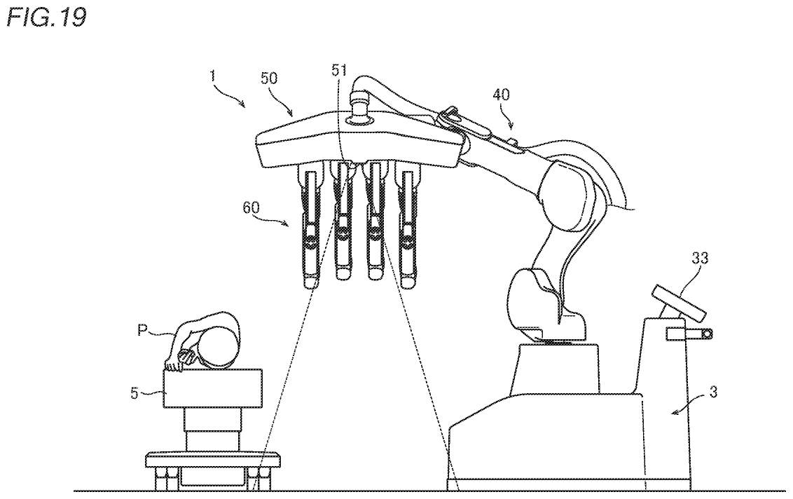

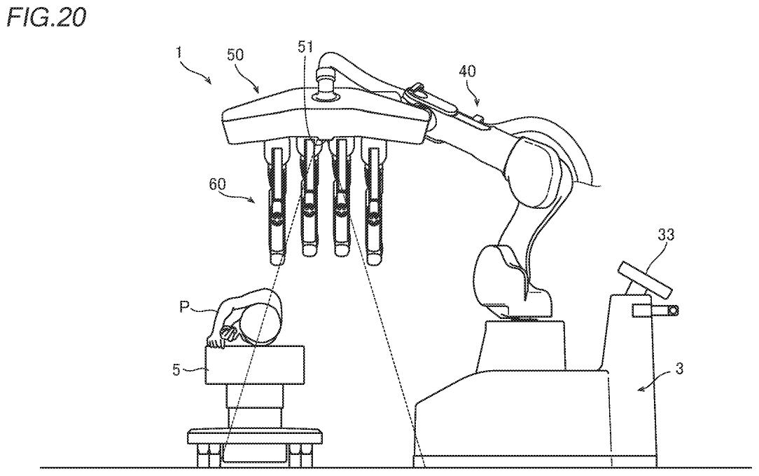

Then, in step S3, as shown in FIG. 18, after the movement operation of the manipulator arms 60, a screen of the display 33a is switched to an image captured by the imager 51. At least one of the surgical table 5 and the patient P placed on the surgical table 5 is imaged by the imager 51 provided on the arm base 50. Specifically, when a distance between the imager 51 of the medical manipulator 1 and the patient P is relatively large, the imager 51 images the surgical table 5 (see FIG. 19). Furthermore, the imager 51 continuously captures images.

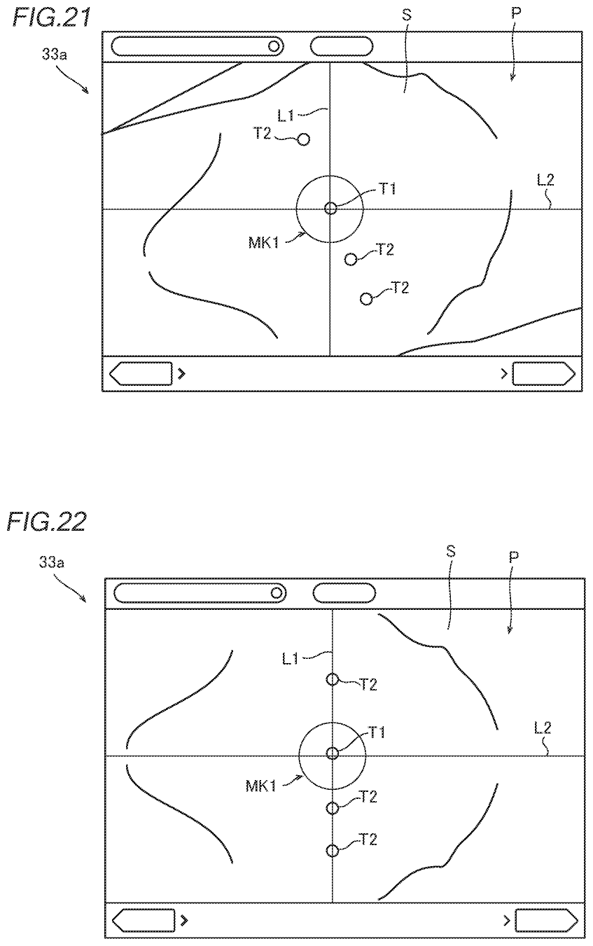

In the first embodiment, then, in step S4, the controller 31 moves the medical cart 3 to the vicinity of the patient P placed on the surgical table 5 based on the image of the surgical table 5 captured by the imager 51, as shown in FIGS. 19 and 20. Specifically, the controller 31 recognizes the surgical table 5 in the image captured by the imager 51 using image recognition technology, and drives the servomotors M5 and M6 to move the medical cart 3 such that the medical cart 3 approaches the surgical table 5. Thus, the ports PT or the trocars T provided on the body surface S of the patient P placed on the surgical table 5 are imaged by the imager 51. Then, the controller 31 recognizes the ports PT or the trocars T in the image captured by the imager 51 using image recognition technology, and moves the medical cart 3 such that the port PT for the endoscope 6 or the trocar T for the endoscope 6 is arranged directly below the imager 51. Thus, the port PT or the trocar T is arranged directly below the imager 51. Then, on the display 33a, the first trocar T1 is arranged inside the substantially circular first mark MK1 (see FIG. 21).

In the first embodiment, then, in step S5, the controller 31 causes the positioner 40 to move the arm base 50 to move the manipulator arms 60 so as to align the manipulator arms 60 with the ports PT or the trocars T based on the image of the ports PT or the trocars T provided on the body surface S of the patient P captured by the imager 51, as shown in FIG. 22. Thus, on the display 33a, the plurality of second trocars T2 are arranged substantially on the first line L1 or the second line L2 of the second mark MK2 with the first trocar T1 arranged inside the substantially circular first mark MK1.

Then, in step S6, the controller 31 shifts each of the plurality of manipulator arms 60 to a setup posture. The setup posture is different from the roll-in posture (the posture in which each manipulator arm 60 is folded), and refers to a posture in which a distance between the manipulator arms 60 is widened such that the pivot position teaching instrument 7 (see FIG. 8) or the endoscope 6 (see FIG. 7) can be easily attached to each of the plurality of manipulator arms 60. The operator such as a nurse or a technician attaches the pivot position teaching instrument 7 or the endoscope 6 to each of the plurality of manipulator arms 60.

Then, in step S7, the controller 31 acquires temporary pivot positions PP based on the arrangement positions of the ports PT. The temporary pivot positions PP are input in advance by the operator. In the first embodiment, the controller 31 acquires the coordinates of the ports PT or the trocars T provided on the body surface S of the patient P and imaged by the imager 51. Specifically, the controller 31 acquires the coordinates of the ports PT or the trocars T by calculating a distance to the ports PT or the trocars T and a distance between the ports PT or between the trocars T based on a plurality of images captured by the imager 51 with different distances between the imager 51 and the patient P. The distance to the ports PT or the trocars T refers a distance between the imager 51 and the body surface S of the patient P.

Then, the controller 31 associates the positional relationship between the tip end of the pivot position teaching instrument 7 or the endoscope 6 attached to each manipulator arm 60 and the temporary pivot position PP based on the calculated distances. Then, the controller 31 recalculates distances between the tip end of the pivot position teaching instrument 7 or the endoscope 6 and the ports PT or the trocars T. Thus, the three-dimensional coordinates of the ports PT or the trocars T are acquired.

In the first embodiment, then, in step S8, the controller 31 moves the manipulator arms 60 based on the acquired coordinates of the ports PT or the trocars T. Specifically, the controller 31 moves the surgical instrument 4 or the pivot position teaching instrument 7 for teaching the pivot position PP to the vicinity of the pivot position PP that serves as a fulcrum for movement of the surgical instrument 4 attached to the manipulator arm 60 based on the acquired coordinates of the ports PT or the trocars T. More specifically, the controller 31 moves the manipulator arm 60 until the surgical instrument 4 or the pivot position teaching instrument 7 contacts the body surface S of the patient P. The controller 31 determines whether or not the surgical instrument 4 or the pivot position teaching instrument 7 has contacted the body surface S of the patient P based on a force applied to the manipulator arm 60 (servomotors M1), a torque sensor (not shown), or current feedback from the servomotors M1, for example.

Then, in step S9, the controller 31 receives teaching of the pivot position PP by the operator while the surgical instrument 4 is in contact with the body surface S of the patient P.

### Advantages of First Embodiment

According to the first embodiment, the following advantages are achieved.

### Advantages of Medical Manipulator

According to the first embodiment, as described above, the controller 31 is configured or programmed to perform at least one of a control to move the medical cart 3 by the servomotors M5 and a control to move the manipulator arms 60 by the arm base 50, to align the manipulator arms 60 with the positions corresponding to the ports PT or the trocars T, which are provided on the body surface S of the patient P placed on the surgical table 5 and into which the surgical instruments 4 are to be inserted, based on the image captured by the imager 51. Accordingly, the manipulator arms 60 are automatically aligned with the positions corresponding to the ports PT or the trocars T by at least one of the control to move the medical cart 3 by the servomotors M5 and the control to move the manipulator arms 60 by the arm base 50, and thus when the medical manipulator 1 is aligned, the burden on the operator can be reduced.

According to the first embodiment, as described above, the controller 31 is configured or programmed to move the medical cart 3 to the vicinity of the patient P placed on the surgical table 5, and move the manipulator arms 60 by the arm base 50 to align the manipulator arms 60 with the positions corresponding to the ports PT or the trocars T based on the image captured by the imager 51. Accordingly, both moving the medical cart 3 to the vicinity of the patient P placed on the surgical table 5 and aligning the manipulator arms 60 with the positions corresponding to the ports PT or the trocars T are automatically performed, and thus when the medical manipulator 1 is aligned, the burden on the operator can be further reduced.

According to the first embodiment, as described above, the controller 31 is configured or programmed to move the medical cart 3 to the vicinity of the patient P placed on the surgical table 5 based on the image of the surgical table 5 captured by the imager 51. Accordingly, even when the ports PT or the trocars T do not appear in the image captured by the imager 51, the medical cart 3 can be easily moved to the vicinity of the patient P placed on the surgical table 5 based on the image of the surgical table 5 captured by the imager 51.

According to the first embodiment, as described above, the controller 31 is configured or programmed to perform at least one of a control to move the medical cart 3 and a control to move the manipulator arms 60 by the arm base 50, to align the manipulator arms 60 with the ports PT or the trocars T based on the image of at least one of the surgical table 5 and the ports PT or the trocars T provided on the body surface S of the patient P, captured by the imager 51. Accordingly, the controller 31 can easily perform at least one of a control to move the medical cart 3 and a control to move the manipulator arms 60 by the arm base 50, to align the manipulator arms 60 with the ports PT or the trocars T by recognizing at least one of the surgical table 5 and the ports PT or the trocars T provided on the body surface S of the patient P, which appear in the image.

According to the first embodiment, as described above, the plurality of manipulator arms 60 are provided and attached to the arm base 50, and the controller 31 is configured or programmed to align the manipulator arms 60 with the positions corresponding to the ports PT or the trocars T, which are provided on the body surface S of the patient P placed on the surgical table 5 and into which the surgical instruments 4 are to be inserted, by moving the arm base 50 based on the image captured by the imager 51. Accordingly, even when the manipulator arms 60 are not appropriately arranged at the positions corresponding to the ports PT or the trocars T by only moving the medical cart 3, the manipulator arms 60 can be appropriately arranged at the positions corresponding to the ports PT or the trocars T by moving the arm base 50.

According to the first embodiment, as described above, the imager 51 is provided on the arm base 50. Accordingly, the relative position of the imager 51 with respect to the arm base 50 does not change, and thus unlike a case in which the relative position of the imager 51 with respect to the arm base 50 changes, a control to move the medical cart 3 can be easily performed based on the image captured by the imager 51.

According to the first embodiment, as described above, the controller 31 is configured or programmed to acquire the coordinates of the ports PT or the trocars T provided on the body surface S of the patient P and imaged by the imager 51, and move the surgical instrument 4 or the pivot position teaching instrument 7 to teach the pivot position PP to the vicinity of the pivot position PP that serves as a fulcrum for movement of the surgical instrument 4 attached to the manipulator arm 60 based on the acquired coordinates of the ports PT or the trocars T. Accordingly, the operator does not need to move the surgical instrument 4 or the pivot position teaching instrument 7 to the vicinity of the pivot position PP when teaching the pivot position PP, and thus the operator's labor can be saved.

According to the first embodiment, as described above, the controller 31 is configured or programmed to acquire the coordinates of the ports PT or the trocars T by calculating the distance to the ports PT or the trocars T and the distance between the ports PT or between the trocars T based on the plurality of images captured by the imager 51 with the different distances between the imager 51 and the patient P. Accordingly, unlike a case in which a separate sensor or the like is provided to calculate the distance to the ports PT or the trocars T, the complexity of the configuration of the medical manipulator 1 can be reduced or prevented.

### Advantages of Control Method for Medical Manipulator

According to the first embodiment, as described above, the control method for the medical manipulator 1 includes performing at least one of a control to move the medical cart 3 by the servomotors M5 and a control to move the manipulator arms 60 by the arm base 50, to align the manipulator arms 60 with the positions corresponding to the ports PT or the trocars T, which are provided on the body surface S of the patient P placed on the surgical table 5 and into which the surgical instruments 4 are to be inserted, based on the image captured by the imager 51. Accordingly, the manipulator arms 60 are automatically aligned with the positions corresponding to the ports PT or the trocars T by performing at least one of the control to move the medical cart 3 by the servomotors M5 and the control to move the manipulator arms 60 by the arm base 50, and thus it is possible to provide the control method for the medical manipulator 1 capable of reducing the burden on the operator when the medical manipulator 1 is aligned.

### Second Embodiment

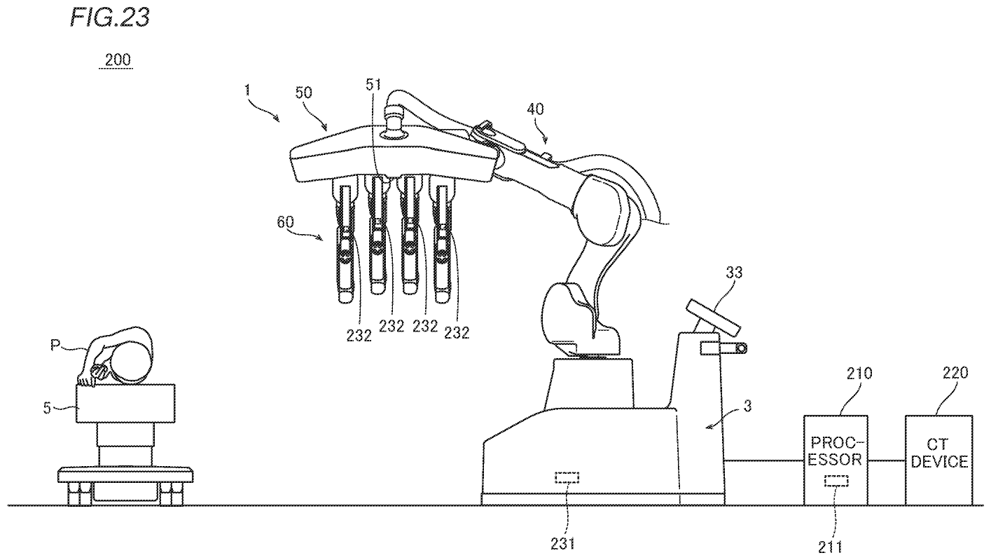

The configuration of a surgical system 200 according to a second embodiment is now described with reference to FIGS. 23 to 26.

As shown in FIG. 23, the surgical system 200 includes a medical manipulator 1 and a processor 210. The configuration of the medical manipulator 1 is the same as or similar to the configuration of the medical manipulator 1 according to the first embodiment.

The processor 210 plans the positions of manipulator arms 60 with respect to positions corresponding to ports PT, which are provided on the body surface S of a patient P placed on a surgical table 5 and into which surgical instruments 4 are to be inserted. Specifically, the processor 210 prepares a three-dimensional model of the patient P from images of the patient P captured in advance, determines the positions of the ports PT into which the surgical instruments 4 are to be inserted based on the three-dimensional model, and plans the positions of the manipulator arms 60 based on the determined positions of the ports PT. A method for planning the positions of the manipulator arms 60 is now described.

### Method for Planning Positions of Manipulator Arms

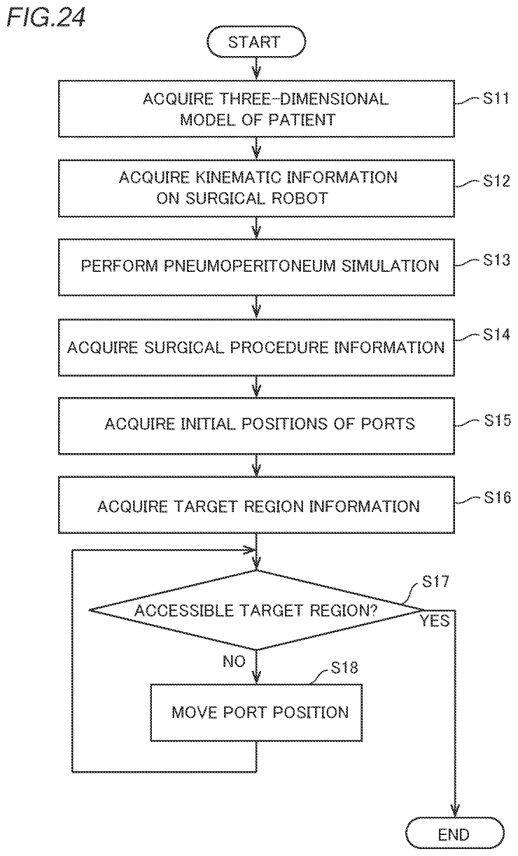

As shown in FIG. 24, in step S11, the processor 210 acquires the three-dimensional model (volume data) generated from the images of the patient P captured by a CT device 220, for example.

Then, in step S12, the processor 210 acquires kinematic information on the medical manipulator 1 from the medical manipulator 1. The kinematic information includes shape information about the shapes of the manipulator arms 60 and the surgical instruments 4 and action information about the actions of the manipulator arms 60 and the surgical instruments 4, for example. This shape information includes the length and weight of each portion of the manipulator arms 60 and the surgical instruments 4, the angles of the manipulator arms 60 with respect to a reference direction (a horizontal plane, for example), and the mounting angles of the surgical instruments 4 with respect to the manipulator arms 60, for example.

Then, in step S13, the processor 210 virtually performs a pneumoperitoneum simulation on the patient P. Specifically, the processor 210 performs a pneumoperitoneum simulation based on pre-pneumoperitoneum volume data acquired from the CT device 220, and generates three-dimensional data as post-pneumoperitoneum volume data.

Then, in step S14, the processor 210 acquires information on the surgical procedure. The treatment to be performed by the medical manipulator 1 is determined by the surgical procedure. Moreover, the surgical instruments 4 necessary for the treatment are determined according to the treatment.

Then, in step S15, the processor 210 acquires the initial positions of a plurality of ports PT according to the acquired surgical procedure. In this case, the processor 210 acquires the three-dimensional coordinates of the initial position of the ports PT. Information on the ports PT includes identification information of the ports PT, information on the positions of the ports PT to be punctured on the body surface S of the patient P, and information on the sizes of the ports PT, for example. The information on the plurality of ports PT is held as a template in a memory 211 within the processor 210 or in an external server. The information on the plurality of ports PT is determined by the surgical procedure, for example.

Then, in step S16, the processor 210 acquires information on a target region from the medical manipulator 1. The target region is a target to be treated by the medical manipulator 1, and includes tissues such as blood vessels, bronchi, organs, bones, brains, hearts, legs, and necks, for example.

Then, in step S17, the processor 210 determines whether or not the surgical instrument 4 inserted through each port PT can access the target region based on the initial positions of the ports PT and the position of the target region. In other words, the processor 210 determines whether or not the medical manipulator 1 can perform the treatment by the surgical instrument 4 according to the acquired surgical procedure.

Then, in a case of NO in step S17, the processor 210 moves at least one of the positions of the plurality of ports PT along the body surface S of the patient P in step S18. The positions of the ports PT may be moved by an operator's input. Then, the process returns to step S17.

Then, in a case of YES in step S17, the processor 210 terminates a process to calculate a plan for the positions of the manipulator arms 60 (port position simulation).

### Planning of Movement Path

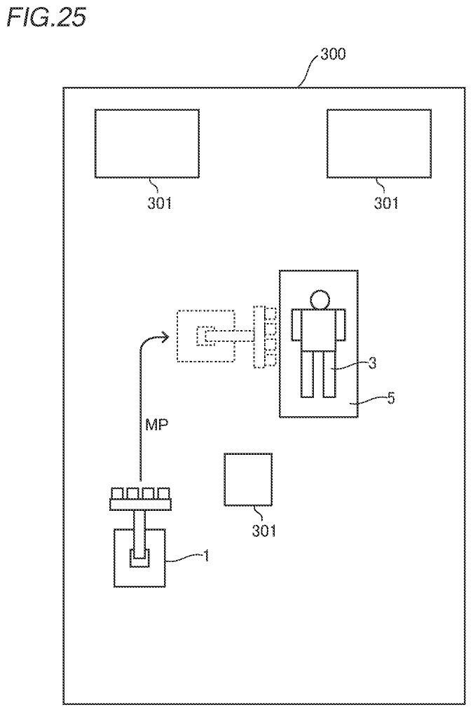

Planning of a movement path MP along which the medical manipulator 1 moves is now described. As shown in FIG. 25, the movement path MP refers to a path along which the medical manipulator 1 moves within an operating room 300.

First, the processor 210 acquires an operating room map showing the planar shape of the operating room 300. The processor 210 also acquires device range areas indicating ranges of devices 301 arranged in the operating room 300. In addition, the processor 210 acquires location areas indicating ranges in which doctors, nurses, and technicians, for example, are located in the operating room 300. The operating room map, the device range areas, and the location areas may be stored in advance in the memory 211 of the processor 210, or may be acquired by the processor 210 from the external server or the like.

Then, the processor 210 calculates the movement path MP based on the operating room map, the device range areas, and the location areas. For example, the movement path MP is calculated such that in the operating room 300, the medical manipulator 1 moves from a location at which the medical manipulator 1 is arranged in advance to the vicinity of the surgical table 5 while avoiding the device range areas and the location areas.

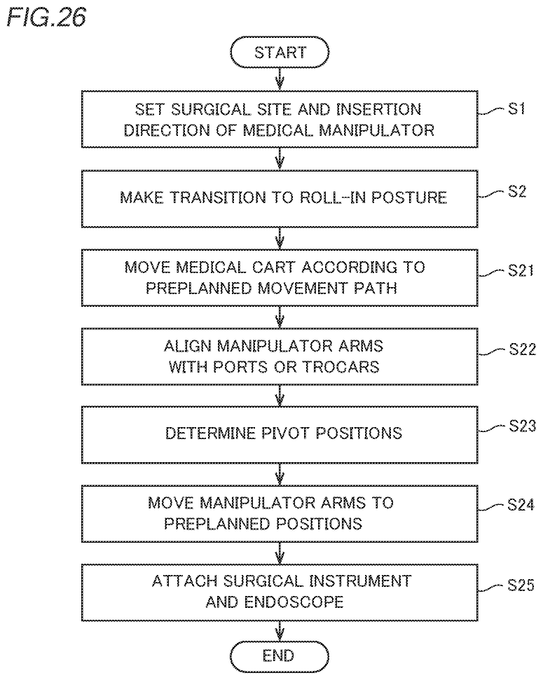

A control method for the medical manipulator 1 is now described with reference to FIG. 26. In the second embodiment, the ports PT or trocars T may be or may not be provided in advance on the body surface S of the patient P placed on the surgical table 5.

The operations in step S1 and step S2 are the same as those of the first embodiment.

In step S21, a controller 231 moves a medical cart 3 according to the preplanned movement path MP. The preplanned movement path MP is acquired from the processor 210. Specifically, in this embodiment, the controller 231 moves the medical cart 3 to the vicinity of the patient P placed on the surgical table 5 by servomotors M5 such that the manipulator arms 60 are arranged at the planned positions based on an image captured by an imager 51. More specifically, the controller 231 moves the medical cart 3 while confirming the current position of the medical manipulator 1 and obstacles based on the image captured by the imager 51. Thus, the ports PT or the trocars T are arranged directly below the imager 51.

Then, in step S22, in the second embodiment, the manipulator arms 60 are moved by the arm base 50 such that the manipulator arms 60 are arranged at the planned positions based on the image captured by the imager 51. Specifically, the manipulator arms 60 are moved by the arm base 50 such that the contour of the patient P in the image captured by the imager 51 matches the contour of the patient P based on the volume data of the patient P used in the port position simulation. Thus, the manipulator arms 60 are aligned with the positions of the ports PT obtained by the port position simulation.

Then, in step S23, the controller 231 automatically determines pivot positions PP based on the preplanned positions of the manipulator arms 60.

Then, in step S24, the controller 231 moves a plurality of manipulator arms 60 to the preplanned positions. That is, the controller 231 shifts each of the plurality of manipulator arms 60 to a setup posture.

Then, in step S25, the operator such as a nurse or a technician attaches the surgical instrument 4 or an endoscope 6 to each of the plurality of manipulator arms 60.

### Advantages of Second Embodiment

According to the second embodiment, the following advantages are achieved.

According to the second embodiment, the controller 231 is configured or programmed to acquire the image captured by the imager 51, and perform at least one of a control to move the medical cart 3 by the servomotors M5 and a control to move the manipulator arms 60 by the arm base 50, to arrange the manipulator arms 60 at the planned positions based on the image. Accordingly, the manipulator arms 60 are automatically placed at the positions planned with respect to the positions corresponding to the ports PT, and thus the burden on the operator can be reduced when the medical manipulator 1 is aligned.

According to the second embodiment, as described above, the controller 231 is configured or programmed to move the medical cart 3 to the vicinity of the patient P placed on the surgical table 5 based on the preplanned movement path MP. Accordingly, even when the ports PT or the trocars T do not appear in the image captured by the imager 51, the medical cart 3 can be easily moved to the vicinity of the patient P placed on the surgical table 5 based on the preplanned movement path MP.

According to the second embodiment, as described above, the controller 231 is configured or programmed to perform at least one of a control to move the medical cart 3 and a control to move the manipulator arms 60 by the arm base 50, to align the manipulator arms 60 with the positions of the ports PT based on the positions of the manipulator arms 60 planned with respect to the positions corresponding to the ports PT, which are provided on the body surface S of the patient P placed on the surgical table 5 and into which the surgical instruments 4 are to be inserted, and the image captured by the imager 51. Accordingly, the manipulator arms 60 can be automatically aligned with the positions corresponding to the ports PT or the trocars T without actually forming the ports PT in the patient P or placing the trocars T.

According to the second embodiment, as described above, the processor 210 is provided to prepare the three-dimensional model of the patient P from the images of the patient P captured in advance and plan the positions of the ports PT based on the three-dimensional model, and the controller 231 is configured or programmed to perform at least one of a control to move the medical cart 3 and a control to move the manipulator arms 60 by the arm base 50, to align the manipulator arms 60 with the ports PT based on the positions of the ports PT determined by the processor 210 and the image captured by the imager 51. Accordingly, the positions of the ports PT are determined based on the three-dimensional model corresponding to the body shape of each patient P, and thus the manipulator arms 60 can be appropriately aligned according to each of the patients P having different body shapes.

According to the second embodiment, as described above, the processor 210 is configured to prepare the three-dimensional model of the patient P from the images of the patient P captured in advance, and plan the positions of the manipulator arms 60 based on the three-dimensional model. Accordingly, the positions of the manipulator arms 60 are planned based on the three-dimensional model corresponding to the body shape of each patient P, and thus the manipulator arms 60 can be appropriately aligned according to each of the patients P having different body shapes.

### Modified Examples

The embodiments disclosed this time must be considered as illustrative in all points and not restrictive. The scope of the present disclosure is not shown by the above description of the embodiments but by the scope of claims for patent, and all modifications (modified examples) within the meaning and scope equivalent to the scope of claims for patent are further included.

For example, while the example in which three second trocars T2 are inserted into the patient P has been shown in each of the aforementioned first and second embodiments, the present disclosure is not limited to this. The number of second trocars T2 may be two, for example.

While the example in which four manipulator arms 60 are provided has been shown in each of the aforementioned first and second embodiments, the present disclosure is not limited to this. The number of manipulator arms 60 may be arbitrary as long as at least one manipulator arm 60 is provided.

While the example in which each of the arm portion 61 and the positioner 40 includes a 7-axis articulated robot has been shown in each of the aforementioned first and second embodiments, the present disclosure is not limited to this. For example, each of the manipulator arms 60 and the positioner 40 may include an articulated robot having an axis configuration (six axes or eight axes, for example) other than the 7-axis articulated robot.

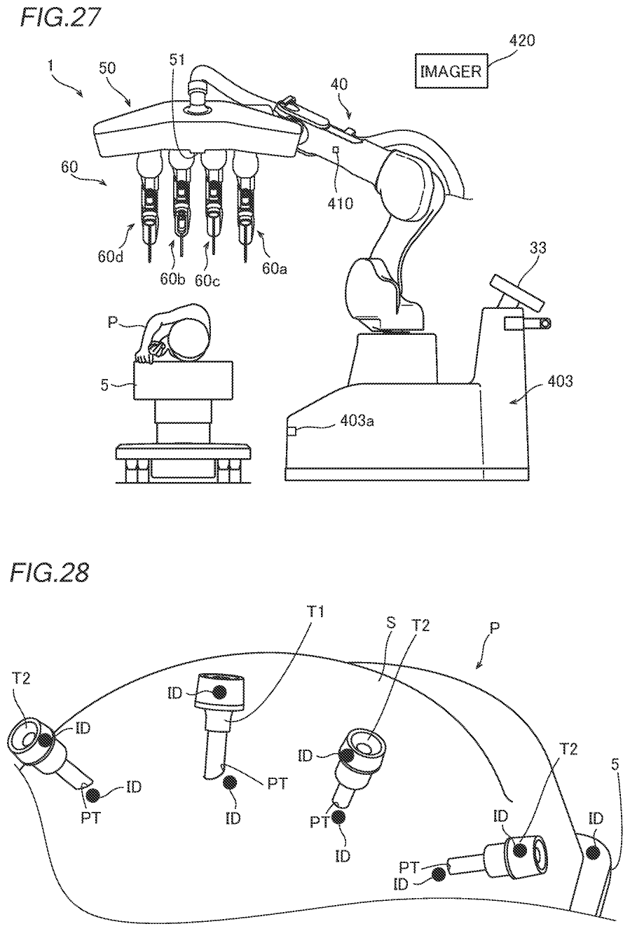

While the example in which the medical cart 3 is moved to the vicinity of the patient P placed on the surgical table 5 based on the image of the surgical table 5 captured by the imager 51 and the ports PT or the trocars T has been shown in each of the aforementioned first and second embodiments, the present disclosure is not limited to this. For example, like a medical cart 403 shown in FIG. 27, the medical cart 3 may be moved to the vicinity of a patient P based on a distance measured by a distance measurement unit 403a provided in the medical cart 403 to measure a distance between a surgical table 5 and the medical cart 403. Alternatively, the medical cart 3 may be moved while the current position of the medical manipulator 1 and obstacles are confirmed based on images captured by an imager 410 provided on a positioner 40 and an imager 420 installed in advance in an operating room 300.

While the example in which the controller 31 (231) moves the medical cart 3 to align the manipulator arms 60 with the ports PT or the trocars T based on the image of the surgical table 5 and the ports PT or the trocars T provided on the body surface S of the patient P, captured by the imager 51 has been shown in each of the aforementioned first and second embodiments, the present disclosure is not limited to this. For example, as shown in FIG. 28, the controller 31 (231) may move the medical cart 3 to align the manipulator arms 60 with the ports PT or the trocars T based on an image of identifiers ID attached to at least one of the surgical table 5 and the ports PT or the trocars T provided on the body surface S of the patient P, captured by the imager 51. The identifiers ID are marks (stickers). Furthermore, indicators (not shown), IC tags, or radio frequency identifiers (RFID) may be used as the identifiers ID, for example. Thus, as compared with a case in which at least one of the surgical table 5 and the ports PT or the trocars T, which may differ in shape or size, for example, are recognized, the common identifiers ID is more easily recognized, and thus at least one of a control to move the medical cart 3 and a control to move the manipulator arms 60 by the arm base 50 can be more easily performed.

While the example in which the controller 31 (231) moves the medical cart 3 such that the ports PT for the endoscopes 6 or the trocars T for the endoscopes 6 are arranged directly below the imager 51 has been shown in each of the aforementioned first and second embodiments, the present disclosure is not limited to this. For example, the controller 31 (231) may move the medical cart 3 such that the ports PT or the trocars T for the endoscopes 6 are arranged at locations slightly spaced apart from a region directly below the imager 51.

While the example in which the controller 31 moves the arm base 50 to align the manipulator arms 60 with the ports PT or the trocars T based on the image of the ports PT or the trocars T captured by the imager 51 has been shown in the aforementioned first embodiment, the present disclosure is not limited to this. For example, the arm base 50 may be moved (rotated) based on data input to the controller 31 in advance, such as the arrangement of the ports PT, or the surgical site and the insertion directions of the surgical instruments 4, instead of using the image of the ports PT or the trocars T captured by the imager 51.

While the example in which the distance to the ports PT or the trocars T and the distance between the ports PT or between the trocars T are calculated in step S8 has been shown in the aforementioned first embodiment, the present disclosure is not limited to this. For example, these distances may be calculated in step S6 of adjusting the positions of the manipulator arms 60 by moving the arm base 50.

While the example in which the controller 31 calculates the distance to the ports PT or the trocars T and the distance between the ports PT or between the trocars T based on the plurality of images captured by the imager 51 has been shown in the aforementioned first embodiment, the present disclosure is not limited to this. For example, a 3D camera or a range sensor may be used instead of the plurality of images. Alternatively, the sizes of the surgical table 5 and the patient P and the distance between the ports PT may be input in advance, and these distances may be calculated by simulation.

While the example in which teaching of the pivot position PP is received while the endoscope 6 or the pivot position teaching instrument 7 attached to the manipulator arm 60 is in contact with the body surface S of the patient P has been shown in the aforementioned first embodiment, the present disclosure is not limited to this. For example, the controller 31 may acquire the coordinates of the ports PT or the trocars T provided on the body surface S of the patient P and imaged by the imager 51, and move the endoscope 6 or the pivot position teaching instrument 7 to a position not contacting the ports PT or the trocars T in the vicinity of the ports PT or the trocars T based on the acquired coordinates of the ports PT or the trocars T.

While the example in which the arm base 50 is moved to align the manipulator arms 60 with the positions corresponding to the ports PT or the trocars T after the medical cart 3 is moved to the vicinity of the patient P placed on the surgical table 5 has been shown in each of the aforementioned first and second embodiments, the present disclosure is not limited to this. For example, the arm base 50 may be moved in advance before the medical cart 3 is moved to the vicinity of the patient P, and the medical cart 3 may be moved to the vicinity of the patient P such that the manipulator arms 60 are aligned with the positions corresponding to the ports PT or the trocars T.

While the example in which the controller 231 is provided in the medical manipulator 1 has been shown in the aforementioned second embodiment, the present disclosure is not limited to this. In the present disclosure, the controller 231 may be provided in the processor 210 other than the medical manipulator 1, for example.

While the example in which a control to move the manipulator arms 60 by the arm base 50 is performed after a control to move the medical cart 3 by the servomotors M5 is performed has been shown in each of the aforementioned first and second embodiments, the present disclosure is not limited to this. For example, the manipulator arms 60 may be aligned only by a control to move the manipulator arms 60 by the arm base 50.

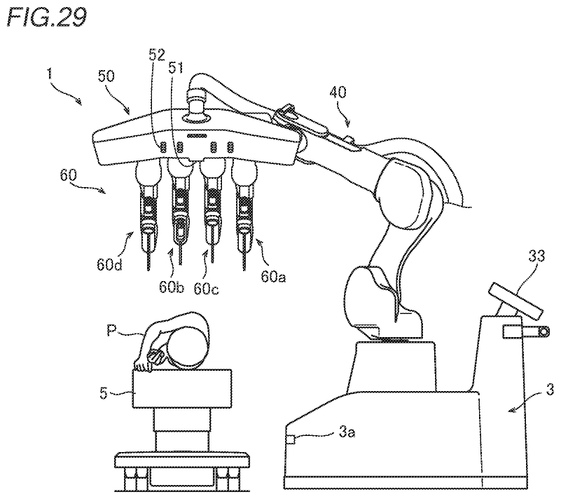

In each of the aforementioned first and second embodiments, as shown in FIG. 29, an obstacle detection sensor 3a may be provided on the medical cart 3, and when moving the medical cart 3 to the vicinity of the patient P placed on the surgical table 5 in step S4 (step S21 in the second embodiment), the controller 31 may move the medical cart 3 backward when the obstacle detection sensor 3a detects an obstacle, and may resume the movement of the medical cart 3 to the vicinity of the patient P. When the obstacle detection sensor 3a detects an obstacle even when the medical cart 3 is moved again, the medical cart 3 may be stopped, and an indicator 52 provided on the arm base 50, for example, may notify the operator such as a nurse or a technician in order to prompt the operator to (manually) move the medical cart 3.

Thus, the obstacle detection sensor 3a detects an obstacle, and thus collision between the medical cart 3 and the obstacle can be reduced or prevented. Furthermore, when the obstacle detection sensor 3a detects an obstacle, the medical cart 3 is moved backward, and the movement of the medical cart 3 to the vicinity of the patient P is resumed such that the medical cart 3 can be moved to the vicinity of the patient P placed on the surgical table 5 while collision between the medical cart 3 and the obstacle is reduced or prevented even in the presence of the obstacle.

The obstacle detection sensor 3a is a contact type sensor or a non-contact type optical sensor, for example. When the obstacle detection sensor 3a is a contact type sensor, the obstacle detection sensor 3a surrounds the medical cart 3. When the obstacle detection sensor 3a is an optical sensor, the obstacle detection sensor 3a is provided on the front surface, side surfaces, and rear surface of the medical cart 3, for example.

When the plurality of manipulator arms 60 are moved to the preplanned positions in step S24 in the second embodiment, the imager 51 or contact sensors provided on the manipulator arms 60 may predict (or detect) contact between the manipulator arms 60 and the patient P or the surgical instrument 4. When contact with the patient P or the surgical instrument 4 is predicted (or detected), movement of the manipulator arms 60 is stopped. Then, the controller 231 performs a control to prompt the operator to manually move the manipulator arms 60 (causes the indicator 52 in FIG. 29 to notify the operator, for example).

### DESCRIPTION OF REFERENCE NUMERALS

1: medical manipulator (surgical robot)
3: medical cart
3a: obstacle detection sensor
4: surgical instrument
5: surgical table
7: pivot position teaching instrument
31, 231: controller
50: arm base (robot main body)
51: imager
60: manipulator arm
210: processor
ID: identifier
MP: movement path
M5, M6: servomotor (medical cart drive)
P: patient
PP: pivot position
PT: port
S: body surface
T: trocar

1. A surgical robot comprising:
a robot main body including a manipulator arm to which a surgical instrument is attached;
a medical cart to move the robot main body;
a medical cart drive to drive the medical cart;
an imager provided on the robot main body to image at least one of a surgical table and a patient placed on the surgical table; and
a controller configured or programmed to perform at least one of a control to move the medical cart by the medical cart drive and a control to move the manipulator arm by the robot main body, to align the manipulator arm with a position corresponding to a port or a trocar, which is provided on a body surface of the patient placed on the surgical table and into which the surgical instrument is to be inserted, based on an image captured by the imager.

2. The surgical robot according to claim 1, wherein the controller is configured or programmed to move the medical cart to a vicinity of the patient placed on the surgical table, and move the manipulator arm by the robot main body to align the manipulator arm with the position corresponding to the port or the trocar based on the image captured by the imager.

3. The surgical robot according to claim 2, wherein the controller is configured or programmed to move the medical cart to the vicinity of the patient placed on the surgical table based on an image of the surgical table captured by the imager or a preplanned movement path.

4. The surgical robot according to claim 2, further comprising:
an obstacle detection sensor provided on the medical cart to detect an obstacle that hinders movement of the medical cart; wherein
the controller is configured or programmed to:
move the medical cart backward when the obstacle detection sensor detects the obstacle; and
resume movement of the medical cart to the vicinity of the patient or stop movement of the medical cart.

5. The surgical robot according to claim 1, wherein the controller is configured or programmed to perform at least one of the control to move the medical cart and the control to move the manipulator arm by the robot main body, to align the manipulator arm with the port or the trocar based on an image of at least one of the surgical table and the port or the trocar provided on the body surface of the patient, captured by the imager.

6. The surgical robot according to claim 5, wherein the controller is configured or programmed to perform at least one of the control to move the medical cart and the control to move the manipulator arm by the robot main body, to align the manipulator arm with the port or the trocar based on an image of an identifier attached to at least one of the surgical table and the port or the trocar provided on the body surface of the patient, captured by the imager.

7. The surgical robot according to claim 1, wherein the controller is configured or programmed to perform at least one of the control to move the medical cart and the control to move the manipulator arm by the robot main body, to align the manipulator arm with a position of the port based on a position of the manipulator arm planned with respect to the position corresponding to the port, which is provided on the body surface of the patient placed on the surgical table and into which the surgical instrument is to be inserted, and the image captured by the imager.

8. The surgical robot according to claim 7, further comprising:
a processor to prepare a three-dimensional model of the patient from images of the patient captured in advance and plan the position of the port based on the three-dimensional model; wherein
the controller is configured or programmed to perform at least one of the control to move the medical cart and the control to move the manipulator arm by the robot main body, to align the manipulator arm with the port based on the position of the port determined by the processor and the image captured by the imager.

9. The surgical robot according to claim 1, wherein
the manipulator arm includes a plurality of manipulator arms;
the robot main body includes an arm base to which the plurality of manipulator arms are attached; and
the controller is configured or programmed to align one of the manipulator arms with the position corresponding to the port or the trocar, which is provided on the body surface of the patient placed on the surgical table and into which the surgical instrument is to be inserted, by moving the arm base based on the image captured by the imager.

10. The surgical robot according to claim 9, wherein the imager is provided on the arm base.

11. The surgical robot according to claim 1, wherein the controller is configured or programmed to:
acquire coordinates of the port or the trocar provided on the body surface of the patient and imaged by the imager; and
move, to a vicinity of a pivot position that serves as a fulcrum for movement of the surgical instrument attached to the manipulator arm, the surgical instrument or a pivot position teaching instrument to teach the pivot position based on the acquired coordinates of the port or the trocar.

12. The surgical robot according to claim 11, wherein the controller is configured or programmed to acquire the coordinates of the port or the trocar by calculating a distance to the port or the trocar and a distance between the ports or between the trocars based on a plurality of images captured by the imager with different distances between the imager and the patient.

13. A robotic surgical system to support robotic surgery using a surgical robot, the robotic surgical system comprising:
the surgical robot, a processor, and a controller; wherein
the surgical robot includes:
a robot main body including a manipulator arm to which a surgical instrument is attached;
a medical cart to move the robot main body;
a medical cart drive to drive the medical cart; and
an imager provided on the robot main body to image at least one of a surgical table and a patient placed on the surgical table;
the processor is operable to plan a position of the manipulator arm with respect to a position corresponding to a port, which is provided on a body surface of the patient placed on the surgical table and into which the surgical instrument is to be inserted; and
the controller is configured or programmed to:
acquire an image captured by the imager; and
perform at least one of a control to move the medical cart by the medical cart drive and a control to move the manipulator arm by the robot main body, to arrange the manipulator arm at the planned position based on the image.

14. The robotic surgical system according to claim 13, wherein the processor is operable to prepare a three-dimensional model of the patient from images of the patient captured in advance, and plan the position of the manipulator arm based on the three-dimensional model.

15. The robotic surgical system according to claim 14, wherein the processor is operable to determine a position of the port into which the surgical instrument is to be inserted based on the three-dimensional model, and plan the position of the manipulator arm based on the determined position of the port.

16. A control method for a surgical robot, the surgical robot including a robot main body including a manipulator arm to which a surgical instrument is attached, a medical cart to move the robot main body, and a medical cart drive to drive the medical cart, the control method comprising:
imaging at least one of a surgical table and a patient placed on the surgical table by an imager provided on the robot main body; and
performing at least one of a control to move the medical cart by the medical cart drive and a control to move the manipulator arm by the robot main body, to align the manipulator arm with a position corresponding to a port or a trocar, which is provided on a body surface of the patient placed on the surgical table and into which the surgical instrument is to be inserted, based on an image captured by the imager.

17. The control method for the surgical robot according to claim 16, further comprising:
moving the medical cart to a vicinity of the patient placed on the surgical table.

18. The control method for the surgical robot according to claim 17, wherein
the moving the medical cart to the vicinity of the patient includes moving the medical cart to the vicinity of the patient placed on the surgical table based on an image of the surgical table captured by the imager or a preplanned movement path.

19. The control method for the surgical robot according to claim 17, the surgical robot further including an obstacle detection sensor provided on the medical cart to detect an obstacle that hinders movement of the medical cart, the control method further comprising:
moving the medical cart backward when the obstacle detection sensor detects the obstacle; and
resuming movement of the medical cart to the vicinity of the patient or stopping movement of the medical cart.

20. The control method for the surgical robot according to claim 16, wherein
the performing at least one of the control to move the medical cart by the medical cart drive and the control to move the manipulator arm by the robot main body includes performing at least one of the control to move the medical cart and the control to move the manipulator arm by the robot main body, to align the manipulator arm with the port or the trocar based on an image of at least one of the surgical table and the port or the trocar provided on the body surface of the patient, captured by the imager.
